(19) United States
(12) Patent Application Publication
(10) Pub. No.: US 2022/0240861 A1
(43) Pub. Date: Aug. 4, 2022

(54) SYSTEMS AND METHODS FOR SPO2 CLASSIFICATION USING SMARTPHONES

(71) Applicant: University of Washington, Seattle, WA (US)

(72) Inventors: Jason Hoffman, Seattle, WA (US); Shwetak N. Patel, Seattle, WA (US); Edward Wang, Seattle, WA (US); Varun Viswanath, Seattle, WA (US)

(73) Assignees: University of Washington, Seattle, WA (US); The Regents of the University of California, Oakland, CA (US)

(21) Appl. No.: 17/164,745

(22) Filed: Feb. 1, 2021

(51) Int. Cl. A61B 5/00; H04M 1/21; H04N 5/225; H04N 9/68; G06T 7/00; G06T 7/90; A61B 5/1455; A61B 5/103

(52) U.S. Cl. CPC A61B 5/6898; H04W 88/02; H04N 5/2256; H04N 9/68; H04N 5/2257; G06T 7/0012; G06T 7/90; A61B 5/14552; A61B 5/7267; A61B 5/7278; A61B 5/1032; A61B 5/6826; A61B 5/7275; A61B 5/4884; H04M 1/21

(57) ABSTRACT

Examples of systems and methods for classifying SpO2 levels using smartphones are described. A wideband light source (e.g., a flash) may be used to illuminate a finger. A wideband imaging sensor (e.g., a camera) may be used to capture images of the illuminated finger. The smartphone may apply per-color channel gain adjustments to the captured images. The adjusted pixel data may be used as the basis of input to a classifier (e.g., a deep learning model). The classifier may be trained on ground truth data, such as from an induced hypoxia study. The classifier may output an SpO2 level blood in the finger.

### TECHNICAL FIELD

Examples described herein relate generally to measurement of SpO2 levels. Examples of SpO2 measurement using a smartphone camera and flash and a machine learning model are described.

### BACKGROUND

Blood-oxygen saturation, reported as Sp02 percentage (e.g., SpO2 level), is the clinical measure that informs a physician of the ability of the body to distribute oxygen by revealing the proportion of hemoglobin in the blood currently carrying oxygen. While a healthy SpO2 level is different for each individual, everybody needs an adequate supply of oxygen in their tissues. Respiratory illnesses such as asthma, Chronic Obstructive Pulmonary Disease (COPD), and COVID-19 can cause significant declines in SpO2, recurrent hypoxemia, and subsequent hypoxia, and serious health complications, such as organ damage, brain damage, and death, can occur if SpO2 stays low for an extended period of time. Recently, in COVID-19 patients, in-hospital mortality rate has been shown to increase when a patient's SpO2 level cannot be maintained above 90%, a level that has also been used in primary care to indicate the need to consult a physician for further care. Frequent measurements of SpO2 can allow for identification of the severity of asthma and COPD, predict mortality amongst COVID-19 patients, and detect presence of other illnesses including Idiopathic Pulmonary Fibrosis, Congestive Heart Failure, Diabetic Ketoacidosis, and pulmonary embolism.

Pulse oximetry for monitoring blood oxygen saturation may be performed through a variety of techniques, including direct arterial blood analysis and purpose-built devices for the detection of specific wavelengths of light. Perhaps the ‘gold standard’ for measuring oxygen saturation is the Arterial Blood Gas analysis device, which takes a blood sample to measure the amounts of oxygenated and deoxygenated hemoglobin. As this technique is too invasive and expensive for most use cases, clinics primarily rely on optical pulse oximeters, which take noninvasive readings of SpO2.

Clinical pulse oximeters typically perform oxygenation measurement via transmittance photoplethysmography (PPG) sensing at the finger tip, clamping around the end of the finger a finger clip device, which measures the light absorption properties through the tissue of the finger to infer blood composition. The clip includes a light source and photodiode sensors on opposite sides of the finger to measure and calculate the light absorption of the pulsatile blood in the finger. The same measurement has been demonstrated, and made available for clinical use, on the toes, ear lobe, and forehead. The measurement at the forehead differs from the others in that it performs reflectance measurements, in which the emitter and receiver are on the same side of the device, relying on the reflectance of some portion of the light from different layers of the tissues, such as the walls of blood vessels. These monitors are used to assess and monitor patients in clinical checkups, in-clinic patient monitoring, and monitoring during surgery.

### BRIEF DESCRIPTION OF THE DRAWINGS

FIG. 1 is a schematic illustration of a smartphone arranged in accordance with examples described herein;

FIG. 2 is a schematic illustration of a smartphone in use during reflectance photoplethysmography (PPG);

FIG. 3 depicts visualizations of color data from a device with standard color gain settings and using custom settings in accordance with examples described herein.

FIG. 4 is a schematic illustration of data processing arranged in accordance with examples described herein.

FIG. 5 is a schematic illustration of classification techniques arranged in accordance with examples described herein.

FIG. 6 is a schematic illustration of the training and operation of a classifier in accordance with examples described herein.

### DETAILED DESCRIPTION

Hypoxemia, a medical condition that occurs when the blood is not carrying enough oxygen to adequately supply the tissues, is a leading indicator for dangerous complications of respiratory diseases like asthma, COPD, and COVID-19. While purpose-built pulse oximeters can provide accurate SpO2 readings that allow for diagnosis of hypoxemia, enabling this SpO2 sensing capability in smartphone cameras could give more people access to important information about their health, as well as improve their physicians' ability to remotely diagnose and treat respiratory conditions. Herein are described examples of smartphone-based SpO2 sensing system which may use a varied inspired fractional oxygen (FiO2) protocol, creating a clinically relevant validation dataset for smartphone-based methods on a large range of SpO2 values (e.g., 65%-100%). Previous systems were generally only evaluated on smaller ranges (e.g., 85%-100%). Examples of deep learning models are described which may be built (e.g., trained) using this data to demonstrate accurate reporting of SpO2 level with an overall MAE<5.0% and identifying positive cases of low SpO2 with >93% recall rate in some implemented examples.

Monitoring SpO2 with a smartphone, particularly an unmodified smartphone, if provided in an accurate and unobtrusive manner, may improve health outcomes for those with respiratory illnesses by providing access to rapid risk assessment outside the clinic. Smartphone-based SpO2 monitors may offer the ubiquity and precision necessary to increase access to detection and treatment of respiratory diseases. Examples described herein provide smartphone-based SpO2 monitors which may operate on a full range of clinically-relevant SpO2 values, such as from 65%-100%.

Currently, clinicians measure SpO2 levels using FDA-cleared, purpose-built devices called pulse oximeters during regular clinic visits, which allows them to asses a patient's condition and evaluate how that condition has changed since a prior visit. While purpose-built pulse oximeters are accurate, non-invasive, and robust across skin colors and SpO2 levels, they possess undesirable characteristics that inhibit use outside the clinic. Users need to (1) purchase the device and (2) have the device with them whenever they need monitoring. These factors reduce the accessibility of more frequent and widespread SpO2 measurements, as patients can forget their devices, fail to charge them, or misplace them, if they can even afford them in the first place. These observations reveal a significant gap in respiratory monitoring, in which sudden, undetected, and dangerous deterioration can occur. This possibility has become more clear in the context of the COVID-19 pandemic, during which it has been shown that hypoxemia can be present in potentially dangerous but otherwise asymptomatic patients.

Smartphone-based SpO2 monitors present the opportunity to detect and monitor respiratory conditions in contexts where pulse oximeters may be inaccessible. Smartphones are widely owned because of their multi-purpose utility, and contain increasingly powerful sensors, including a camera with a LED flash. Some existing smartphone-based SpO2 sensing only present proof-of-concept studies which may produce data in a limited range of 85%-100% SpO2 through techniques like breath-holding. Lower SpO2 percentages may be more difficult to measure using commodity hardware and also more expensive to collect. Nonetheless, to realize ubiquitous SpO2 sensing with smartphones, it may be desirable for the smartphone-based SpO2 monitors to meet the standards for data breadth to which current pulse oximeters are held.

Examples described herein include smartphone-based SpO2 monitoring system which may be designed, built, and validated with a balanced dataset that covers a clinically relevant breadth of SpO2 validation. Data used for validation was collected by delivering controlled medical grade oxygen-nitrogen mixtures of varied Fractional Inspired Oxygen (FiO2) levels to subjects while they were monitored by both an example smartphone device as described herein and a traditional pulse oximeter. This type of study and dataset allowed for an assessment of techniques described herein on low SpO2 examples (e.g., below 85%).

An implemented analysis on 6 subjects revealed that a convolutional neural network was a able to achieve MAE<5.0 on predicting a new subject's SpO2 level, after it had been trained on 5 other subjects' labeled data, and an average precision to recall tradeoff of 76.0% to 93.2% on classifying a new subject's SpO2 as below 90%.

Certain details are set forth herein to provide an understanding of described embodiments of technology. However, other examples may be practiced without various of these particular details. In some instances, well-known circuits, control signals, timing protocols, and/or software operations have not been shown in detail in order to avoid unnecessarily obscuring the described embodiments. Other embodiments may be utilized, and other changes may be made, without departing from the spirit or scope of the subject matter presented here.

From the foregoing it will be appreciated that, although specific embodiments have been described herein for purposes of illustration, various modifications may be made while remaining with the scope of the claimed technology.

Examples described herein may refer to various components as “coupled” or signals as being “provided to” or “received from” certain components. It is to be understood that in some examples the components are directly coupled one to another, while in other examples the components are coupled with intervening components disposed between them. Similarly, signal may be provided directly to and/or received directly from the recited components without intervening components, but also may be provided to and/or received from the certain components through intervening components.

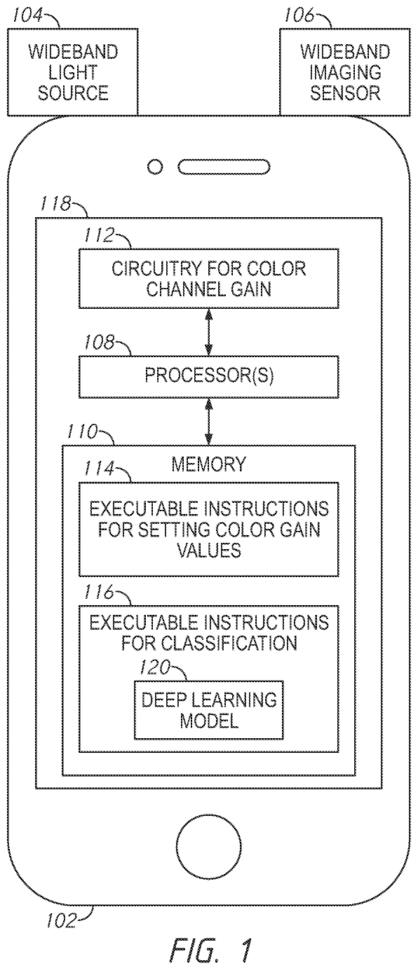

FIG. 1 is a schematic illustration of a smartphone arranged in accordance with examples described herein. The smartphone 102 includes wideband light source 104 and wideband imaging sensor 106. The smartphone 102 includes display 118, circuitry for color channel gain 112, processor(s) 108, and memory 110. The memory 110 includes executable instructions for setting color gain values 114 and executable instructions for classification 116, which may include deep learning model 120. Additional, fewer, and/or different components may be present in other examples. For example, the smartphone 102 may include one or more communication interface(s), networking interface(s), additional memory and/or electronic storage, and/or additional software. The processor(s) 108 may execute instructions stored in memory 110 and/or in other computer readable media accessible to the smartphone 102 and/or processor(s) 108 to perform the setting of color gain values for the smartphone 102 and/or classification of SpO2 levels in blood of users.

Examples of systems described herein may accordingly include smartphones. Smartphone 102 is shown in FIG. 1. Generally, a smartphone may include any consumer electronic device in communication with a wideband light source and/or wideband imaging sensor as described herein and with one or more processors and/or communication interfaces to conduct the classification described herein to predict an SpO2 level of blood as described herein. A smartphone may or may not have cellular phone capability, which capability may be active or inactive. While smartphones are described, examples of techniques described herein may be implemented in some examples using other electronic devices such as, but not limited to, tablets, laptops, computers, appliances, or vehicles. Generally, any device having a light source, imaging sensor, and processor(s) may be used.

Smartphones described herein may come in a variety of models. A model of a smartphone may generally refer to a particular set of hardware components (e.g., flash, camera, processor, etc.) and/or software components (e.g., operating system) used to implement the smartphone. The particulars of these hardware components may vary across smartphone models. Smartphones may also have a make, which may in some examples be included in the model. Examples of models include iPhone 11, iPhone 10, Galaxy S20, Galaxy Note 20, Google Nexus 6P, etc. Other models may also be used. The make of a smartphone may refer to the brand of smartphone (e.g., Samsung, Apple, Nokia, Sony).

Smartphones described herein may include one or more wideband light sources, such as wideband light source 104 of FIG. 1. For example, the wideband light source 104 may be implemented using a flash of the smartphone 102. The wideband light source 104 may be implemented using, for example, one or more light emitting diodes (LEDs). Generally, a wideband light source may emit energy over multiple color wavelengths (e.g., red, green, and blue). These may be referred to as color channels. The wideband light source 104 may be used, e.g., under the control of processor(s) 108 in some examples, to illuminate a finger (including a portion of a finger). The finger, such as a fingertip, may be placed in contact with the wideband light source 104 for illumination in some examples.

Smartphones described herein may include one or more wideband imaging sensors, such as wideband imaging sensor 106. The wideband imaging sensor 106 may be implemented using a camera of the smartphone 102. The wideband imaging sensor 106 may generally refer to a sensor which may be sensitive to incident energy over multiple color wavelengths (e.g., red, green, and blue), which may also be referred to as color channels. The wideband imaging sensor 106 may be used, e.g., under the control of processor(s) 108 in some examples, to capture pixel data. In some examples, pixel data of an illuminated finger (e.g., a fingertip) may be captured by the wideband imaging sensor 106.

The wideband light source 104 and wideband imaging sensor 106 may be positioned in a variety of locations in or on the smartphone 102. In some examples, the wideband light source 104 and/or wideband imaging sensor 106 may not be integral to the smartphone 102 but may be in electronic communication with the smartphone 102. In some examples, the wideband light source 104 and/or wideband imaging sensor 106 may be integral to the smartphone 102. In some examples, the wideband light source 104 and/or wideband imaging sensor 106 may be positioned on a front of the smartphone 102, a back of the smartphone 102, and/or along an edge of the smartphone 102. In some examples, the wideband light source 104 and wideband imaging sensor 106 are positioned proximate one another. For example, the wideband light source 104 and wideband imaging sensor 106 may be positioned such that a finger of a user may contact both the wideband light source 104 and wideband imaging sensor 106.

In some examples, in addition to wideband light source 104 and wideband imaging sensor 106, or incorporated in those components, an infrared (IR) source and sensor may be used. The IR proximity sensor, for example, may provide an avenue to measure the blood's IR absorption property simultaneously. Some example smartphones may include an infrared based focus system that uses a proximity sensor that houses a IR LED and IR optical sensor. By using this pair, which is placed next to the camera and flash, when the finger covers the camera, flash, and IR proximity sensor, the blood's absorption at R/G/B/IR can be measured simultaneously or in an overlapping fashion. The IR channel may be used as another color channel described herein, and a color gain values may be set for that channel as well.

Smartphones described herein may include circuitry for color channel gain 112. The circuitry for color channel gain 112 may include any of a variety of hardware components which manipulate data from the wideband imaging sensor 106 in particular color channels. The circuitry for color channel gain 112 may be coupled to wideband imaging sensor 106. The circuitry for color channel gain 112 may include one or more filters, amplifiers, analog-to-digital converters, and/or logic circuits. The circuitry for color channel gain 112 may selectively operate on particular color channels of data (e.g., red, green, blue). Per-channel color gain values may be specified for each color channel. The circuitry for color channel gain 112 may, for example, include an amplifier associated with each color channel, and that amplifier may have a particular gain value. In some examples, the color gain values may be larger for the blue channel and the green channel than for the red channel. In some examples, the circuitry for color channel gain 112 may apply gain which may vary across particular color channels—e.g., a gain value may be specified for a red channel, a gain value may be separately specified for a blue channel, and a gain value may be separately specified for a green channel. The gain value may be different for each channel in some examples, while in some examples one or more channels may share a same gain value. In some examples, the circuitry for color channel gain 112 may gain-adjust green and blue channels of the pixel data more than a red channel of the pixel data. For example the circuitry for color channel gain 112 may provide more gain-adjustment to the green and blue channels than to the red channel. In one example, the gain value may be 1× for the red channel, 3× for the green channel, and 18× for the blue channel. Other values may also be used. The gain values in some examples may be selected based on the model of the smartphone. For example, particular gain levels may be used based on the hardware and/or software components included in a particular model of smartphone. The gain values in some examples may be selected based on empirical study data. Examples described herein generally provide for setting the per-channel color gain values such that multiple color channels in the pixel data are not clipped—e.g., variation in each of the multiple color channels is available for use in classifying the adjusted pixel data into a predicted SpO2 level.

In some examples, certain parameters of the circuitry for color channel gain 112 may be set in hardware, software (e.g., in accordance with executable instructions for setting color gain values 114), or combinations thereof. The gain of each amplifier may in some examples be set using a software interface. Generally, in examples described herein, the circuitry for color channel gain 112 may adjust pixel data received from the wideband imaging sensor 106 using per-channel color gain values that may be selected to maintain data in the multiple color channels within a digitization threshold. The circuitry for color channel gain 112 may output adjusted pixel data, which may be used by classification software described herein.

Smartphones described herein may include one or more processors, such as processor(s) 108 of FIG. 1. Any number or kind of processing circuitry may be used to implement processor(s) 108 such as, but not limited to, one or more central computing units (CPUs), graphical processing units (GPUs), logic circuitry, field programmable gate arrays (FPGAs), application specific integrated circuits (ASICs), controllers, or microcontrollers. While certain activities described herein may be described as performed by the processor(s) 108 it is to be understood that in some examples, the activities may wholly or partially be performed by one or more other processor(s) which may be in communication with processor(s) 108. That is, the distribution of computing resources may be quite flexible and the smartphone 102 may be in communication with one or more other computing devices, continuously or intermittently, which may perform some or all of the processing operations described herein in some examples.

Smartphones described herein may include memory, such as memory 110 of FIG. 1. While memory 110 is depicted as integral with smartphone 102, in some examples, the memory 110 may be external to smartphone 102 and may be in communication with processor(s) 108 and/or other processors in communication with processor(s) 108. While a single memory 110 is shown in FIG. 1, generally any number of memories may be present and/or used in examples described herein. Examples of memory which may be used include read only memory (ROM), random access memory (RAM), solid state drives, and/or SD cards.

Smartphones described herein may operate in accordance with software (e.g., executable instructions stored on one or more computer readable media, such as memory, and executed by one or more processors). Examples of software may include executable instructions for setting color gain values 114 of FIG. 1. The executable instructions for setting color gain values 114 may provide instructions and/or settings for controlling the circuitry for color channel gain 112 as described herein. For example, the executable instructions for setting color gain values 114 may provide one or more amplifier settings for use in adjusting pixel data for particular color channels. These may be referred to as per-channel color gain values.

Examples of software may include executable instructions for classification 116 of FIG. 1. The executable instructions for classification 116 may provide instructions for predicting an SpO2 level of blood using a machine learning model, such as deep learning model 120 of FIG. 1. Examples described herein may accordingly provide one or more machine learning models, such as deep learning model 120 of FIG. 1. Generally, a machine learning model may refer to a mathematical model which is able to classify input data into a particular outcome. The mathematical model may in some examples be represented as a set of weights and/or connections between nodes in a multi-layered neural network. In some examples, the machine learning model may have been trained on sample data. In some examples, the deep learning model 120 may be trained using data from one or more induced hypoxia studies. The deep learning model 120 may be trained prior to or after being stored in memory 110. In some examples, training of deep learning model 120 may be ongoing during use of smartphone 102. In some examples, the executable instructions for classification 116 may include instructions for predicting the SpO2 level below 85 percent in some examples, below 80 percent in some examples. Generally, the executable instructions for classification 116 may predict SpO2 levels between 85 and 100 percent in some examples, between 80 and 100 percent in some examples, between 75 and 100 percent in some examples, between 70 and 100 percent in some examples, or between 65 and 100 percent in some examples. Other ranges may also be used. In some examples, because the deep learning model 120 had been trained on data from one or more induced hypoxia studies, a resulting prediction may be able to be more accurate, particularly at lower levels, such as below 85 percent. In some examples, the deep learning model 120 may be particular to the smartphone and/or model of smartphone used. For example, a model may be trained based on the response and performance of a particular phone and/or model for phone. The deep learning model 120 loaded on the smartphone 102 may be selected in accordance with the model of the smartphone 102 and/or the particular hardware present on the smartphone 102.

During operation, a finger (such as a fingertip) may be positioned to receive illumination from the wideband light source 104 (e.g., may be placed in contact with the wideband light source 104). The finger may also be positioned to be imaged by the wideband imaging sensor 106, such as by being placed in contact with the wideband imaging sensor 106. The smartphone 102 may, in accordance with the processor(s) 108 executing executable instructions, illuminate the finger with the wideband light source 104, and capture pixel data with the wideband imaging sensor 106. The pixel data from the wideband imaging sensor 106 may be provided to the circuitry for color channel gain 112. The circuitry for color channel gain 112 may gain-adjust the pixel data to provide gain-adjusted pixel data. The manner in which the circuitry for color channel gain 112 may gain-adjust the pixel data may be set, e.g., using executable instructions for setting color gain values 114, in accordance with a model of the smartphone. The smartphone 102 may utilize the executable instructions for classification 116 including deep learning model 120 to predict an SpO2 level of blood in the finger based on the gain-adjusted pixel data.

In some examples, the smartphone 102 may have hardware and/or software to detect excessive motion of the finger and provide feedback (e.g., audio, visual, and/or haptic feedback) to the user to keep still, or keep the finger still and/or discard high motion segments. For example, motion may be detected by the processor(s) 108 analyzing pixel data captured by the wideband imaging sensor 106 for anomalies consistent with a moving finger. If a moving finger was detected, the display could display a reminder to remain still and/or reposition the finger, an audio tone or instruction could be played by the smartphone 102, and/or the smartphone 102 may vibrate.

The predicted SpO2 level may be used in a variety of ways. The SpO2 level may be displayed, e.g., on display 118 of the smartphone 102. The SpO2 level may be sent to another software program operating on smartphone 102 and/or to another computing device from the smartphone 102. SpO2 levels may be monitored using the smartphone 102 at generally any frequency, including continuous and/or semi-continuous monitoring. The SpO2 level may be used to take actions to increase a user's blood oxygen level—for example, a decision to seek further care, provide supplemental oxygen, or take medication may be based on the predicted SpO2 level.

In this manner, examples of smartphones described herein which may predict SpO2 levels may be used as a complete or partial replacement for traditional pulse oximeters by regressing a continuous SpO2 value in some examples.

Examples of smartphones described herein may be used as an at-home screening tool to inform the need for a follow-up with a physician by classifying regression results as below a particular threshold. For example, the smartphone 102 may generate a predicted SpO2 level and generate an alert (e.g., a visual, audio, and/or tactile alert) when the predicted SpO2 level is below a threshold (e.g., 90 percent in some examples).

Note that a smartphone camera and flash may be used to generate data that may be used to predict an SpO2 level in accordance with techniques described herein. In this manner, an unmodified smartphone (e.g., a smartphone without special-purpose attachments or peripherals) may be used to measure SpO2 levels.

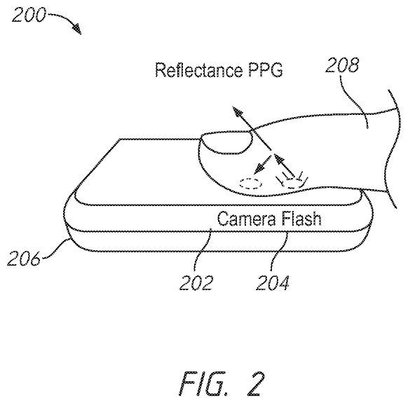

FIG. 2 is a schematic illustration of a smartphone in use during reflectance photoplethysmography (PPG). FIG. 2 depicts smartphone 206, having camera 202 and flash 204. A finger 208 may be placed in contact with camera 202 and flash 204. The components shown in FIG. 2 are exemplary only. Additional, fewer, and/or different components may be used in other examples. The smartphone 102 of FIG. 1 may be implemented by and/or used to implement the smartphone 206 of FIG. 2. The wideband light source 104 of FIG. 1 may be implemented by and/or used to implement the flash 204 of FIG. 2. The wideband imaging sensor 106 of FIG. 1 may be implemented by and/or used to implement the camera 202 of FIG. 2.

The flash 204 may illuminate the finger 208, such as by illuminating a fingertip or other portion of the finger 208. The camera 202 may receive incident energy that is reflected and/or otherwise received from the finger 208 responsive to the illumination. The incident energy received at the camera 202 may be provided as an output of the camera 202 as pixel data. Reflectance PPG techniques may generally be used to obtain SpO2 levels from the pixel data and/or other measurements of the incident energy.

In general, the principle behind reflectance photoplethysmography in a human finger is to evaluate the attenuation, or reduction in intensity, of light through multiple layers of tissue and fluid inside the human finger, and record the pulse-like waveform on a photo-detector. Light is absorbed and reflected differently by different layers of blood, tissue, and bone. The pulse-like waveform of recorded light is characterized by two characteristics: (1) the trough, or DC component, which represents the intensity of light reflected by the static components of the finger, and (2) the trough to peak variance, or AC component, which represents the intensity of light reflected by the time-varying pulsatile blood components. The pulsatile blood components are composed of hemoglobin in two forms, oxyhemoglobin and deoxyhemoglobin, which differ in that oxyhemoglobin is hemoglobin bound to oxygen molecules. Oxygen saturation is determined as the ratio of the concentration of oxyhemoglobin to the concentration of total hemoglobin in the blood, as defined in Equation 1:

$${S}_{p} {O}_{2} = \frac{{ρ}_{O 2}}{{ρ}_{O 2} + {ρ}_{Hb}} , \qquad Equation 1$$

where ρO2 is the concentration of oxyhemoglobin and ρHb is the concentration of deoxyhemoglobin in the blood and is typically reported as a percentage value.

In a healthy adult, it is expected that over 92% of the hemoglobin in arterial blood is carrying oxygen at any given time, though this threshold can vary with pre-existing conditions. To compute this ratio non-invasively, light attenuation can be measured as indicated by the Beer-Lambert Law in Equation 2, which states that light intensity I₀ diminishes exponentially when traveling distance d through a medium with a extinction coefficient at wavelength λ.

I_{measured}=I₀e^{-α[C]d} Equation 2

Because oxyhemoglobin and deoxyhemoglobin have different extinction coefficients, α, at the red (660 nm) and infrared (940 nm) wavelengths, the ratio of the variance in the pulsatile signals at these two wavelengths correlates to oxygen saturation. The DC components in this ratio is used to normalize for the effect of the tissue and other static components on the light. The result from one wavelength may be divided by the other to reveal the absorption ratio in Equation 3:

$${SpO}_{2} = A - B \frac{{AC}_{RED} / {DC}_{RED}}{{AC}_{IR} / {DC}_{IR}} \qquad Equation 3$$

where AC_{RED} and DC_{RED} refer to the AC and DC components, respectively, of signal at the red wavelength; and AC_{1R} and DC_{1R} refer to the AC and DC components, respectively, of signal at the infrared (IR) wavelength. Equation 3 may be used by transmittance pulse oximeters to compute SpO2 after calibrating for different sensor types with a linear fit, but there may be challenges of applying it to reflectance photoplethysmography using a smartphone.

While finger clip pulse oximeters can apply these principles in analyzing the relative attenuation of light on dedicated hardware built to produce and sense narrow-band wavelengths of light at the red and infrared spectra smartphone-based pulse oximetry as described herein may analyze reflected light in only the visible band. In some cases, this may be due to the use of an infrared filter which may typically be included over the smartphone camera in common smartphone hardware. The difference in the extinction coefficients between oxy- and deoxy-hemoglobin in the blue and green bands is not as differentiable as in the infrared band. Also, the wideband light source (e.g., LED) and imaging sensor (e.g., camera) found in smartphones produce and sense light in the visible spectrum. The noisier and less-desirable signal may be the trade-off exchanged for the improved ubiquity and accessibility of a multi-purpose device.

In order to validate a new pulse oximeter system for clinical safety, devices should be tested for accuracy in a study where subjects are given medical grade oxygen-nitrogen mixtures in different levels of varied Fractional Inspired Oxygen (FiO2). The test subjects are expected to have a variety of skin tones. Reflectance and ear-based devices should achieve root mean squared accuracy of <3.5% while transmittance devices should achieve <3% RMS.

Examples described herein utilize data from varied FiO2 experiments which allow for collection of samples in the 65% to 80% SpO2 range. This is physiologically possible because the test subject has time for their body to adjust to breathing in less oxygen at each SpO2 level. At least in part due to this, subject's body is able to tolerate breathing in an oxygen-nitrogen mixture near 70% for an extended period of time. In contrast, breath-holding causes the subjects body to suddenly drop in SpO2 once the subject uses up all the oxygen in the breath that he or she has been holding. When the subject's SpO2 drops to 90%, sometimes lower depending on their health, the subject will physically no longer be able to hold their breath with light-headedness and discomfort. This leads to relatively few samples below 90% SpO2 in data collected from breath-holding experiments.

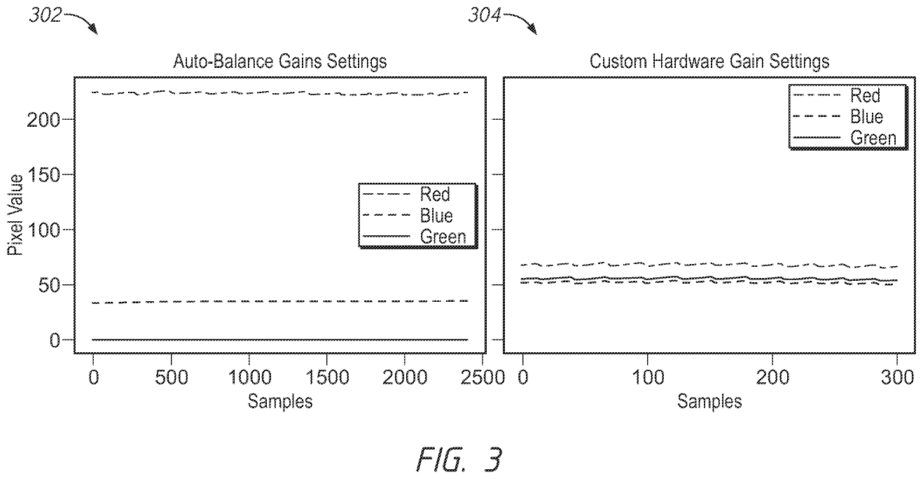

FIG. 3 depicts visualizations of color data from a device with standard color gain settings and using custom settings in accordance with examples described herein. The left-hand graph, graph 302, illustrates a visualization of color data from a device (e.g., a smartphone, such as smartphone 102 of FIG. 1) using standard color gain settings. The right-hand graph, graph 304, illustrates a visualization of color data from a device (e.g., a smartphone, such as smartphone 102 of FIG. 1) using custom hardware gain settings, such as those specified by circuitry for color channel gain 112 in accordance with executable instructions for setting color gain values 114. The graphs illustrate samples (e.g., time) on the x-axis and pixel value (e.g., intensity) on the y-axis. Values for red, green, and blue channels are shown in each graph. The values shown in the graph refer to values of the pixel data after adjustment by the circuitry for color channel gain, such as circuitry for color channel gain 112, described herein. Accordingly, adjusted pixel data is shown in FIG. 3.

In the left image, graph 302, the resolution on green channel is so low that the heartbeat cannot be seen/detected from the green channel data. In the right image, graph 304 however, the pulsation is visible in all three channels. That is, the gain values have been selected such that pulsation is detectible in each color channel. This generally illustrates how standard settings may lead to poorer data quality, and examples described herein that set particular per-channel gain values may improve an ability to predict SpO2 from the adjusted pixel data.

Generally, a camera sensor, such as wideband imaging sensor 106 of FIG. 1, may be exposed based on three factors: exposure time, sensor sensitivity, and aperture. For an RGB camera, all three color channels have the same exposure time and aperture. Both oxygenated and deoxygenated hemoglobin have a much higher absorption coefficient in the blue and green wavelengths than for the red wavelengths by about two orders of magnitude. Thus, it may not be possible to measure all three wavelengths simultaneously under the same exposure. If the hardware sensor's sensitivity to a particular color is too high or too low, pixel values for that color may ‘clip’ by recording the minimum or maximum value of 0 or 255. Because phones use an 8-bit precision scheme for storing pixel data, if the gain is too low for a certain color, the pixels may all be rounded to 0 and small changes in that color will be lost and/or be undetectable. In applications described herein, red may be by far the most dominant color, and with the use of white balance presets for incandescent light, the tones between blue and green may be amplified. So, for example, the circuitry for color channel gain 112 and/or executable instructions for setting color gain values 114 may utilize white balance presets for the wideband imaging sensor 106 to adjust a gain of color channels.

Examples of smartphones may include software which allows for independent control of each color channel's exposure through independent amplifier gain settings (e.g., executable instructions for setting color gain values 114). By having control of independent amplifier gain settings, the exposure settings may be balanced to amplify the blue and green channels more significantly. Different operating systems may allow for a different granularity in the gain control settings. For example, the Android Camera2 API provides access to manual setting of sensitivity, exposure, and individual color gains.

In this manner, rather than relying on a smartphone's auto-balancing feature for the camera and/or allowing the phone to auto-balance itself, exposure parameters may be controlled for SpO2 measurement—e.g., using per-channel color gain values and/or exposure time and aperture selected for SpO2 measurement.

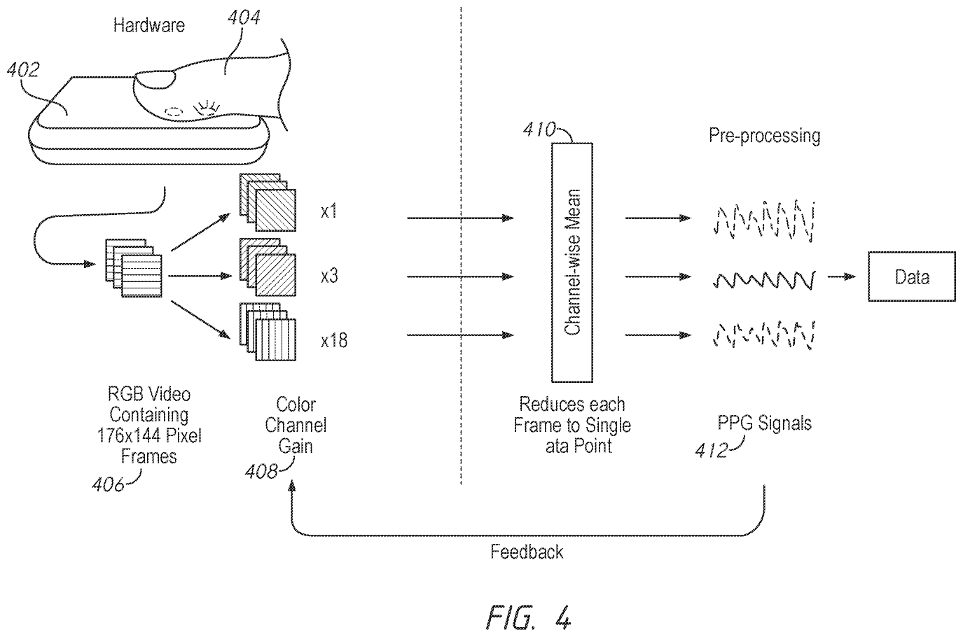

FIG. 4 is a schematic illustration of data processing arranged in accordance with examples described herein. FIG. 4 includes smartphone 402 which may be used to illuminate and record pixel data from finger 404. FIG. 4 illustrates how pixel data may be generated and processed prior to classification. Illumination and recording may provide pixel data 406. The pixel data 406 may be adjusted using color gain values 408. The adjusted pixel values may be provided to pre-processor 410. The pre-processor 410 may perform a variety of pre-processing operations to generate PPG signals 412. The components and techniques described with reference to FIG. 4 are exemplary, and additional, fewer, and/or pre-processing manipulations may be performed in other examples.

The operations discussed with reference to FIG. 4 may be performed by smartphones described herein, such as by smartphone 102 of FIG. 1. For example, the wideband light source 104, wideband imaging sensor 106, processor(s) 108, circuitry for color channel gain 112, and/or software executing on smartphone 102 may be used to implement the pre-processing described and depicted with reference to FIG. 4.

By illuminating finger 404 and imaging the finger 404 responsive to illumination, pixel data 406 may be obtained by the imaging sensor (e.g., wideband imaging sensor 106 of FIG. 1). The pixel data may include a set of image frames (e.g., set of pixel data). Any of a variety of frame rates may be used. In some examples, the pixel data may be captured as a video.

The pixel data may be adjusted by color gain values 408. The color gain values 408 may be implemented by hardware of the smartphone 402, such as by circuitry for color channel gain 112 of FIG. 1. The color gain values may be set per-channel. In the example of FIG. 4, the color channel gain for the red channel is shown as 1—this may refer to the pixel data in the red channel being unmodified by color gain values 408. The color gain value for the green channel is shown as 3. This may refer to the pixel values associated with the green color channel being multiplied by a factor of 3. The color gain value for the blue channel is shown as 18. This may refer to the pixel values associated with the blue color channel being multiplied by a factor of 18. Other gain values may be used in other examples. Gains for the R, G, and B channels may be empirically determined.

In some examples, gains for the R. G. and B channels may be automatically and/or programmatically determined. For example, initial values may be selected, and an output may be examined based on certain metrics—such as a difference between the minimum and maximum value of the signal and an indication of whether the signal has clipped (e.g., hit a highest possible or lowest possible value, such as 0 or 255 in the example using 256 pixel values to encode the data). Gain values may be selected in this manner based on feedback from PPG signals modified by initial gain values. The feedback may be used to modify and/or select gain values, such as by maximizing the minimum-to-maximum signal value for each channel and/or eliminating or reducing clipping. The gains may be selected to avoid and/or reduce clipping or biasing towards one channel.

The adjusted pixel data may receive a variety of pre-processing, such as averaging or smoothing. In the example of FIG. 4, this is shown as being implemented by pre-processor 410. The pre-processor 410 may be implemented using a smartphone, such as smartphone 102 of FIG. 1 or smartphone 402 of FIG. 4. For example, the smartphone 102 may include hardware and/or software for performing the pre-processing described herein. The pre-processing may occur per-color channel. In some examples, an average pixel value for each color channel may be calculated for each frame. In the example of FIG. 4, a data point may be generated for each frame. The data point may in some examples include three values—one for each color channel. This manipulation may generate PPG signals 412. If n frames are taken, the data representing PPG signals may be a 3×n matrix—with 3 values for each frame (one for each color channel). Each frame is represented as an average red channel value, an average green channel value, and an average blue channel value. In some examples, only selected pixels of the frame may be used in calculating the average. In some examples, a weighted average or other combination may be used to generate PPG signals.

In some examples, the PPG signals, and/or metrics based on the PPG signals may be used as feedback to set, change, and/or adjust gain values. For example, the gain values may in some examples be calibrated to generate color gain values which may achieve usable results over a range of lighting conditions. In some examples, an average level of each color channel (e.g., R, G, and B) of the PPG signals 412 may be calculated and used as feedback to adjust the color gain values. For example, the executable instructions for setting color gain values 114 of FIG. 1 may include instructions for setting and/or adjusting color gain values based on feedback, such as an average value of one or more channels in the PPG signals. In some examples, initial color gain values may be those selected by the smart phone in accordance 3 with an auto-balance procedure. The color gain values may be adjusted based on feedback from the output of the auto-balance procedure to attain predetermined goals for the channel values in the PPG signals. Generally, the executable instructions or calibration process may aim to adjust and/or set the color gain values such that the pixels values in each channel are not clipping or saturating and occur within the same range of the color spectrum e.g., (+/−30%). Other tolerances may be used in other examples.

Examples described herein may refer to classification based on adjusted pixel data. It is to be understood that classification based on adjusted pixel data may utilize adjusted pixel data as an input to a classification technique and/or may utilize data which has been pre-processed in some way, such as PPG signals 412.

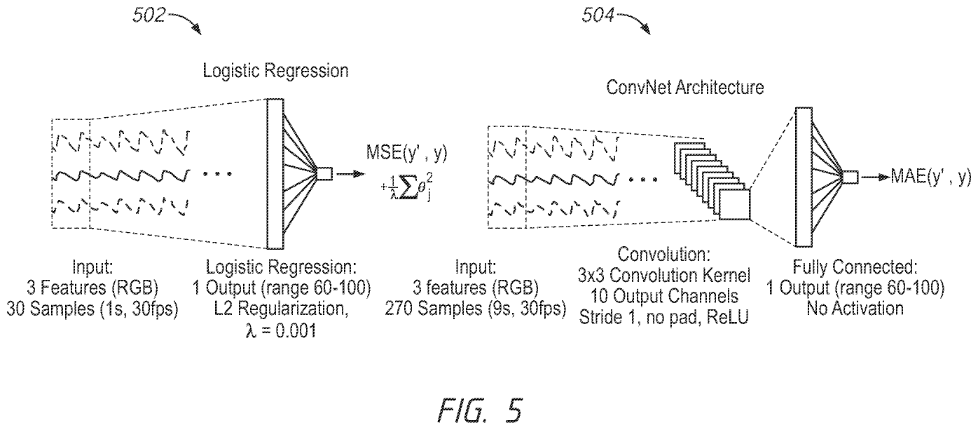

FIG. 5 is a schematic illustration of classification techniques arranged in accordance with examples described herein. Classification techniques may be used to predict SpO2 levels based on adjusted pixel data as described herein (e.g., adjusted pixel data and/or PPG data). Classification may be performed, by example, using smartphone 102 of FIG. 1. The smartphone 102 of FIG. 1 may perform classification in accordance with executable instructions for classification 116, including deep learning model 120. FIG. 5 illustrates two examples of classification techniques—logistic regression 502 and convolutional neural network 504.

Logistic regression 502 may generally refer to a statistical model that uses a logistic function to model a dependent variable. The logistic regression 502 may not use training data that has been normalized across each color channel. Some examples may use the standard deviation of the data (e.g., adjusted pixel data or data based on adjusted pixel data, such as PPG signals) to calculate an AC component of a signal for use in SpO2 classification, but that may not be used in other examples. Logistic regression 502 may use the SK-learn library.

Logistic regression 502 may receive as input 3-channel RGB data computed from multiple frames of pixel data. In the example, of FIG. 5, 30 samples of data are mentioned, representing 1 second each. Other frames and time periods may be used in other examples. The logistic regression 502 may be configured to output a predicted SpO2 level, such as between 60-100 in the example of FIG. 5, although other ranges may be used. The logistic regression 502 may be configured to arrive at the SpO2 level by minimizing L2 loss, and may apply an L2 regularization term to the weights with a power, such as λ=0.001 in the example of FIG. 5 which is shown in Equation 4. In Equation 4, F(X) and f(xᵢ) represent the output of the model on a batch or single sample, Y and yᵢ represent the ground truth for a batch or single sample, θ is the parameters or the weights of the model, and n is the size of the batch.

$$Loss ( F ( X ) , Y ; θ ) = \sqrt{{\sum }_{i = 0}^{n} {( f ( {x}_{i} ) - {y}_{i} )}^{2}} + \frac{1}{λ} {\sum }_{j} {θ}_{j}^{2} \qquad Equation 4$$

In some examples, convolutional neural network 504 may be used. The convolutional neural network 504 may be used instead of logistic regression 502 in some examples. The convolutional neural network 504 may receive adjusted pixel data and/or data based on adjusted pixel data (e.g., PPG signals) as input and provide a predicted SpO2 level as output. The convolutional neural network 504 of FIG. 5 is depicted as receiving 3 features, e.g., three channels (e.g., an R value, G value, and B value) for each of 270 samples, representing 9 seconds of video at 30 frames per second. Other frame rates, sample sizes, and features may be used in other examples. The convolutional neural network 504 may apply a convolution kernel to the input data to produce an predicted SpO2 level as output, such as in a range between 60-100. In another example, three input channels (e.g., R, G, B) may be used for each of 90 frames, which may be taken, for example from 3 seconds of video data taken at 30 frames per second. Other numbers of frames or frame rates may be used in other examples.

The convolutional neural network 504 may be trained on ground truth training data in some examples. Compared to NN-based image recognition tasks, the 1-D, 3-channel RGB data used as input to convolutional neural network 504 may be considered to have low dimensionality. Therefore, a neural network solution (e.g., convolutional neural network 504) may be used with fewer parameters, so as to improve the likelihood of the model's ability to generalize. The convolutional neural network 504 may have a single convolutional layer with a number of output channels (e.g., 10) followed by a dense layer. For the first convolution, the RGB channel components of the input signals may be treated as a second dimension and kernel sizes of 3×3 may be used with no padding. Training and validation data sets may be normalized and standardized based on a weighted channel-wise mean and standard deviation of the training dataset, where the weights may be scaled by the length each subject's data collection. So, if subject 1 was recorded for 12 minutes and subject 2 was recorded for 10 minutes, both subjects would be equally weighted in the training set mean and standard deviation calculation. The model may be trained using the Adam optimizer with a learning rate of 0.01 and an L2 regularization of strength 0.1, although other training techniques may be used. Mean Absolute Error (MAE) may be optimized as a loss function, although other optimization criteria may be used. The convolutional neural network 504 may be built and trained using the PyTorch library. The channel size and size of input window may be chosen through a hyperparameter grid search, although other selection criteria may be used.

Other implementations of a convolutional neural network 504 may also be used. In some examples, the convolutional neural network 504 may be implemented using a deep learning model having 2 convolutional layers and 1 linear layer (e.g., combinations of computations) operating on the input data (e.g., 3 seconds of RGB video data, representing 90 frames for 3 seconds at 30 frames per second).

An output of the convolutional neural network 504 may be a predicted SpO2 level of an individual, which may be evaluated using a mean average error (MAE). The MAE may be compared to ground truth data, such as standalone pulse oximeter readings.

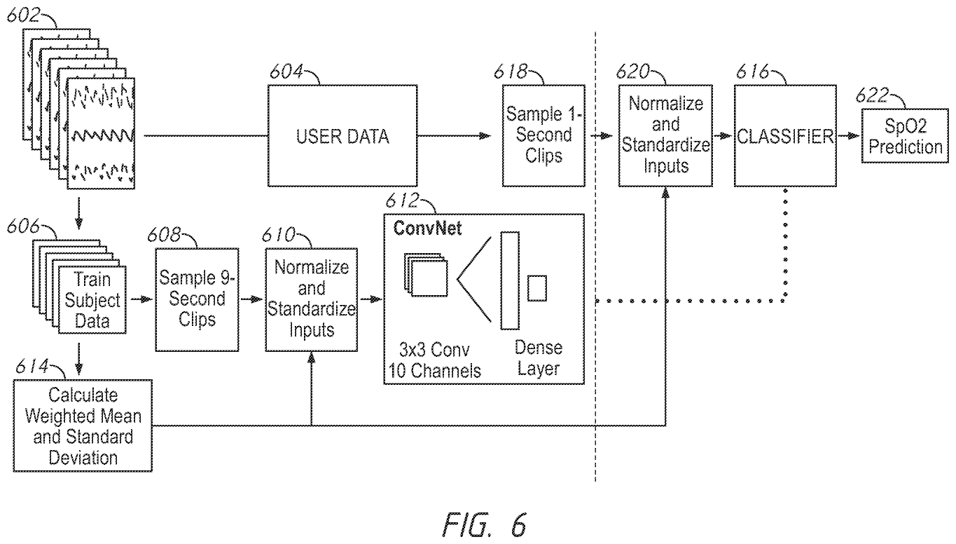

FIG. 6 is a schematic illustration of the training and operation of a classifier in accordance with examples described herein. The classifier 616 may be a trained classifier, and may be implemented using and/or may be used to implement the executable instructions for classification 116 of FIG. 1, together with the processor(s) 108 of FIG. 1 in some examples. Training of the classifier 616 may be performed generally by any computing system. The training may occur prior to use of a smartphone to classify SpO2 levels in some examples. The training may occur to provide a deep learning model, such as deep learning model 120 of FIG. 1, which may be a trained model. In some examples, some or all of the training may be provided by the smartphone itself, such as by smartphone 102. The trained model may then be used to classify user data to predict an SpO2 level associated with the data.

FIG. 6 includes PPG signals 602, some or all of which may be used as training data 606. The training data 606 may, for example, be data from subjects having known SpO2 levels (e.g., from an induced hypoxia study also referred to as an FiO2 study). In the example, there may be five sets of training data, each of which may be from a different subject and/or hand of a subject. Each set of training data may, for example, represent a video of PPG signals where the SpO2 level varies over a range (e.g., 65-100). The training data 606 may be subject to sampling 608 and normalization 610 before being used to train convolutional neural network 612. For example, each set of training data 606 may be sampled in that each set may include data representative of multiple SpO2 levels over time (e.g., from an induced hypoxemia study). Accordingly, a segment of the training data may be sampled which may generally correspond to a single SpO2 level (e.g., 9 seconds of data, or some other amount of time). The convolutional neural network 612 may be implemented using, for example, the convolutional neural network 504 of FIG. 5.

The training data 606 may also be provided to statistic calculator 614 to calculate statistics based on the training data 606, such as weighted mean and standard deviation. For example, statistics may be calculated for each color channel in the training data, such as red, green, and blue color values. In this manner, some statistics about the training data 606 may be generated. The training actions, such as sampling 608, normalization 610, convolutional neural network 612, and/or statistic calculator 614 may be performed by one or more processor(s) which may access a computer readable media and execute instructions for performing the same. The training process may results in a trained classifier, e.g., classifier 616. The training process may ensure, for example that the convolutional neural network 612 may be iteratively updated such that predictions from the neural network model correspond with ground truth SpO2 levels recorded for the training data. Accordingly, weights for the convolutional neural network 612 may be calculated during the training process. Those weights are then shown implemented as classifier 616.

In some examples, such as the “implemented examples” described below, performance of the trained classifier 616 may be evaluated by providing another set of PPG signals 602, not used during the training, as an input to the trained classifier 616. For example, a set of PPG signals 602 not used during the training process may be provided as user data 604. The user data may be sampled 618, and normalized and/or standardized 620 and provided as normalized, standardized inputs to the classifier 616. In some examples, the statistics calculated based on the training data may be used to normalize and/or standardize the new inputs in 620. An SpO2 level predicted by the trained classifier 616 may be compared to any available known ground truth SpO2 data associated with the user data 604 to evaluate the performance of the trained classifier 616, as reported in the “implemented examples” section.

During operation, new user data 604 may be obtained (e.g., by illuminating and imaging a user's finger). The data may not have ground truth SpO2 data associated with it—it may be new subject data for which an SpO2 prediction is desired. The data may be subject to sampling 618 and normalization 620. The normalization 620 may occur with reference to the statistics calculated based on the training data, e.g., in statistic calculator 614. The normalized user data may be provided to classifier 616, which may output an SpO2 prediction 622.

### IMPLEMENTED EXAMPLES

Varied FiO2 Study.

A varied FiO2 study was performed using the varied fractional inspired oxygen protocol administered by a clinical validation laboratory, Clinimark, a group that performs validation services for medical devices. This experiment was approved by the Internal Review Board. Six subjects were administered controlled fractional mixtures of medical grade oxygen-nitrogen in a controlled hospital setting. The subjects rested comfortably in a reclined position while the gas mixture was given to induce hypoxemia in a stair-stepped manner. During this time, the subjects' fingers were instrumented with transmittance pulse oximeter clips and placed on two smartphone devices, with one smartphone device on the index finger of each hand. Ground truth data was recorded using multiple purpose-built pulse oximeters, including a tight-tolerance transfer standard pulse oximeter, the Masimo Radical-7. Subject characteristics and data statistics can be seen in Table 1.

TABLE 1
Subject breakdown for the FiO₂ study.

| | SpO₂ (%) | | | | Duration | | | |
|---|---|---|---|---|---|---|---|---|
| Subject | Mean | Median | Min | Max | (sec.) | Skin Tone | Sex | Age |
| Subject 1 | 87.15 | 91 | 65 | 100 | 1090 | White | Male | 31 |
| Subject 2 | 88.66 | 89 | 73 | 99 | 1121 | Black | Male | 34 |
| Subject 3 | 86.71 | 90 | 66 | 100 | 1066 | White | Female | 23 |
| Subject 4 | 90.29 | 90 | 78 | 100 | 1015 | White | Male | 20 |
| Subject 5 | 85.80 | 87 | 66 | 99 | 926 | White | Female | 24 |
| Subject 6 | 83.86 | 85 | 61 | 99 | 833 | White | Female | 23 |
| Mean/Range | 87.08 | 88.67 | 68.17 | 99.50 | 1008.50 | 1B/5W | 3F/3M | 20-34 |

Ground truth data statistics (in SpO₂ %) for each subject.
The average difference between mean and median for each subject is 1.58, showing little skew.
The average length of each run is about 16 minutes.

Noteworthy observations were also recorded, including the observation that one subject, Subject 1 in the analysis, had particularly callused hands, which we might have interfered with examples described herein.

Smartphone Device Configuration and Setup. Smartphone data was collected with a Google Nexus 6P, recording video at 30 frames per second. The device was specifically configured so that hardware camera settings did not change throughout the entire study. This was done by locking auto-balancing and enhancing color gain, a unique step in this system. The color gains were set to 1× for the red channel, 3× for the green channel, and 18× for the blue channel. These values were chosen based on an empirical study with 20 health individuals the best gain values were manually analyzed to avoid data loss due to compression and obtain optimal signal quality. During the Varied FiO2 study, because the device could overheat from recording continuous video for too long, clay ice packs were placed around the device to keep its temperature down. The ice packs were placed strategically to avoid contact with the hand.

Manual Hardware Sensitivity, Exposure, and Whitebalance Settings. To ensure that the blue and green signals are not lost, a fixed color gain was empirically determined and assigned, ensuring that a usable signal is recorded by the camera for all 3 color channels. The empirically determined gains were 1, 3, 18 for R, G, and B in this example. After setting the color channel, the use of 1.2 ms for exposure time and a sensor sensitivity of 300 ISO was also determined empirically to perform well in evenly exposing R, G, and B color channel PPG signals at the middle of the 0-255 value range.

As shown in FIG. 3, the left side shows an example of directly measuring the PPG signal with standard auto-balancing algorithm. It can be seen that the red PPG clips at the top of the range while the green and blue channels are close to 0. In comparison, using our custom hardware gain settings controlled through the Android Camera2 API, all three color channel PPGs are well represented in the 8 bit range on the right side graph (graph 304) of FIG. 3.

Data Preprocessing. For each hand on each subject, an ordered list of a number, n, of RGB image frames were obtained, each with 176×144 pixels. To obtain a PPG signal, we take the mean pixel value for each color channel and obtain a 3×n shaped matrix of values. Because humans are asymmetrical, different internal arm and body structures can lead to differences in blood flow to the right and left arms. These differences were seen in the data collected. Therefore, each hand subject pair was treated as a unique subject. However, predictions were visualized for the right and left arm adjacently for ease of comparison. Finally, the data was divided into 1 sample for each 1-second (30 frames) window, combining the 3 seconds (90 frames) of sample RGB data centered on 1 ground truth SpO2 reading as one sample. This provides over 5000 training examples (5 subjects) to the models, with about 1000 samples (1 subject) held out for the test set for each round of LOOCV

Pulse Oximetry Validation. A regression analysis was performed to compare smartphone measurements taken in accordance with examples described herein (e.g., with reference to FIG. 1-FIG. 6 to a purpose-built pulse oximeter with error and Bland-Altman metrics. In the performance assessment, models were evaluated using Leave-One-Subject-Out cross validation (LOOCV). Specifically, training and testing were performed on six validation splits, with a different subject (both hands) held out for validation in each split. The ground truth distributions of the splits were visually examined to ensure there is not heavy imbalance in the dataset. The performance of algorithms was compared using Mean Absolute Error.

Hypoxemia Screening Tool. A classification analysis was performed, thresholding the ground truth recordings below 3 different SpO2 levels (95%, 90%, and 85%) and compared to the thresholded regression result. We examine how the true positive and false positive rates at different screening decision boundaries perform to illustrate the potential performance for triaging depending on the needs of a use case. To interrogate the potential to adjust this decision boundary to bias towards recall vs. precision, we vary the decision boundary across the range of 80%-100% and plot ROC curves for each subject using LOOCV.

SpO2 Prediction via Pulse Oximetry

The logistic regression model achieved a mean absolute error of 5.40% with L2 regularization. During development, the introduction of L2 regularization strongly improved the regressor's performance. The 1-layer convolutional neural network model produced a mean average error (MAE) of 4.13 when analyzed via LOOCV against all the data that was gathered in the varied FiO2 study. While this performance is slightly better than that of the logistic regression model, the convolutional network has similar behavior in predictions across different SpO2 values to the logistic regression. The model performed best on Subject 2 achieving a mean absolute error of 2.89%, a mean and std. difference of −2.34% and 4.65%. For this subject, in the SpO2 ranges of 65%-80%, 81%-90%, and 91%-100%, the model achieved mean differences of −0.47%, −3.07%, and −2.53%, and standard deviations of differences of 2.34%, 3.57%, and 5.25%. Excluding subject 1, the model performed worst on Subject 5 achieving a mean absolute error of 4.29%, a mean and std. difference of −0.68% and 10.11%. For this subject, in the SpO2 ranges of 65%-80%, 81%-90%, and 91%-100%, the model achieved mean differences of 3.75%, −4.31%, and −1.06%, and standard deviations of differences of 9.97%, 7.0%, and 6.34%.

The mean difference and standard deviation of difference statistics in each ground truth range, as well as overall, are summarzied in Table 2. The average mean difference across all 6 subjects on all ground truth values is −0.77.

TABLE 2

[symbol]

| | | | μ_{d}/LOA | μ_{d}/LOA | μ_{d}/LOA |
|---|---|---|---|---|---|
| Subject | MAE | μ_{d}/LOA | (65%-80%) | (81%-90%) | (91%-100%) |
| 1 | 6.46 | 4.52/15.02 | 15.35/7.27 | 5.2/3.65 | −1.72/3.43 |
| 2 | 2.89 | −2.34/4.65 | −0.47/2.34 | −3.07/3.57 | −2.53/5.25 |
| 3 | 3.57 | 1.66/9.7 | 6.91/9.3 | −1.43/8.41 | 0.3/3.31 |
| 4 | 3.87 | −1.69/7.73 | 5.08/2.3 | −1.21/8.68 | −3.14/4.25 |
| 5 | 4.29 | −0.68/10.11 | 3.75/9.97 | −4.31/7.0 | −1.06/6.34 |
| 6 | 3.68 | 3.16/6.85 | 5.36/7.5 | 2.4/3.63 | 1.37/5.81 |
| Average | 4.13 | 0.77/9.01 | 6.0/6.45 | −0.4/5.82 | −1.13/4.73 |

[symbol]

[symbol]

indicates data missing or illegible when filed

When we separate examples into ground truth ranges, 65%-80%, 81%-90%, and 91%-100%, respectively, the average mean differences across subjects in each range are 6.0, −0.4, −1.13. Further, for Subjects 3, 5, and 6, from 65%-80%, there was a negative trend in predictions and the mean difference is above the limits of agreement for some ground truth values. Accordingly, the model shows a pattern of consistently over-predicting on SpO2 samples below 80% in this example. It is worth noting that without the varied FiO2 study such as the one we carried out, we may not observe the model performance below 85% at all.

To explore the potential of using the example smartphone camera oximeter system as a screening tool for hypoxemia, the classification accuracy of an example model was calculated in providing an accurate indication about whether an individual has an SpO2 level below three different thresholds: 95%, 90%, and 85%. A reading below 90% SpO2 is a common threshold below which it is recommended to reach out for immediate medical attention, but other thresholds could be clinically useful to screen for different individuals based on their condition. Thus, the ability of an example system to classify samples from the test set by thresholding the regression result from a CNN at different decision boundaries and comparing it to whether the ground truth pulse oximeter also reads less than that value. Precision and recall was computed across all combinations of LOOCV to compute an average result. This simulates the device screening a subject it has never seen before, as the model was trained only on the other 5 subjects in our dataset.

The results of this classification algorithm can be seen in Table 3. For the 90% classification problem, the model correctly classifies 93% of the samples that were below 90% (recall) in the data set, while 76% of its overall classifications were correct (precision). For 95%, those numbers go up to 97% recall with 86% precision, on average across all 6 test subjects.

TABLE 3

| Subject | <95% (P/R) | <90% (P/R) | P/R < 85% (P/R) |
|---|---|---|---|
| 1 | .76/.93 | .65/.89 | .93/.68 |
| 2 | .81/1.0 | .80/.99 | .76/.66 |
| 3 | .92/.99 | .66/.95 | .74/.92 |
| 4 | .85/1.0 | .67/.94 | .24/.45 |
| 5 | .88/.99 | .80/.93 | .67/.61 |
| 6 | .92/.92 | .99/.91 | 1.0/.89 |
| Mean | .86/.97 | .76/.93 | .72/.70 |

Not all combinations of test and train subjects displayed the same level of accuracy. In order to visualize classification accuracy across our entire dataset, the classification threshold was varied for each classification goal between 80%-100% and the results averaged across all 6 combinations of LOOCV. For <90% classification, the highest precision to recall ratio was 0.76 to 0.93 with an F-score of 0.84, when using a regression decision threshold of 91. This means that the model classified a truly low SpO2 level correctly 93% of the time with this threshold, while being correct on 76% of all of the classification decisions. However, it may be preferable to choose a threshold that enables higher recall to bias the system towards classifying low SpO2 cases correctly more often. For example, with the current model and validation data, choosing a decision threshold of 93 on the regression result allowed for greater than 98% accuracy at identifying positive cases (ground truth <90% SpO2), while only resulting in 29% false positives.

What is claimed is:

1. A method comprising:
illuminating a finger with a wideband light source including wavelengths in multiple color channels;
imaging the finger with a wideband imaging sensor to obtain pixel data;
adjusting the pixel data using per-channel color gain values configured to maintain data in the multiple color channels within a digitization threshold, to provide adjusted pixel data; and
classifying the adjusted pixel data using a deep learning model to predict an SpO₂ level of blood in the finger.

2. The method of claim 1, wherein said illuminating comprises using a flash of a smartphone, and said imaging comprises using a camera of a smartphone, and wherein said per-channel color gain values are particular to a model of the smartphone.

3. The method of claim 1, wherein the multiple color channels comprise a red channel, a green channel, and a blue channel, and wherein the color gain values are different for each of the red channel, the green channel, and the blue channel.

4. The method of claim 3, wherein the color gain values are larger for the blue channel and the green channel than for the red channel.

5. The method of claim 3, wherein the color gain values are selected based on empirical study data.

6. The method of claim 1, wherein the color gain values are selected based on feedback from signals generated using initial color gain values.

7. The method of claim 1, wherein the deep learning model is trained using data from an induced hypoxemia study.

8. The method of claim 1, wherein said classifying is configured to predict an SpO₂ level lower than 85 percent.

9. A non-transitory computer readable media encoded with instructions which, when executed by a processor cause a system to perform actions comprising:
gain-adjust pixel data received from a smartphone camera, the pixel data corresponding to an illuminated finger, such that multiple color channels in the pixel data are not clipped;
provide the gain-adjusted pixel data to a deep learning model; and
predict an SpO₂ level of blood in the finger using the deep learning model.

10. The non-transitory computer readable media of claim 9, wherein the deep learning model is trained using data from an induced hypoxia study.

11. The non-transitory computer readable media of claim 9, wherein said gain-adjust pixel data comprises applying a gain adjustment particular to a model of the smartphone.

12. The non-transitory computer readable media of claim 9, wherein the predict an SpO₂ level is accurate below 85 percent SpO₂.

13. The non-transitory computer readable media of claim 9, wherein the multiple color channels comprise a red channel, a green channel, and a blue channel, and wherein the green channel and the blue channel are adjusted more than the red channel.

14. The non-transitory computer readable media of claim 9, wherein the pixel data corresponds to the finger illuminated using a flash of the smartphone.

15. A smartphone comprising:
a flash;
a camera;
a processor;
memory encoded with executable instructions which, when executed by the processor cause the smartphone to:
illuminate a finger with the flash;
capture pixel data with the camera;
gain-adjust the pixel data in accordance with a model of the smartphone; and
predict an SpO₂ level of blood in the finger based on the gain-adjusted pixel data using a deep learning model.

16. The smartphone of claim 15, wherein the deep learning model is trained using data from an induced hypoxia study.

17. The smartphone of claim 15, wherein the flash comprises a wideband light source.

18. The smartphone of claim 15, wherein the camera comprises a wideband imaging sensor.

19. The smartphone of claim 15, wherein the deep learning model is configured to predict the SpO₂ level below 85 percent.

20. The smartphone of claim 15, wherein the executable instructions further cause the smartphone to gain-adjust green and blue channels of the pixel data more than a red channel of the pixel data.
